SEQUENCE LISTING (2 sequences)

SEQ ID NO: 1 - DNA, 53 bp, synthetic construct
  source 1..53: mol_type = other DNA; note = Primer Sequence
  variation 36: note = W is A or T/U
tcgtcggcagcgtcagatgtgtataagagacagacwcctacgggwggcagcag

SEQ ID NO: 2 - DNA, 51 bp, synthetic construct
  source 1..51: mol_type = other DNA; note = Primer Sequence
gtctcgtgggctcggagatgtgtataagagacagattaccgcggctgctgg

(19) United States
(12) Patent Application Publication
(10) Pub. No.: US 2024/0016862 A1
(43) Pub. Date: Jan. 18, 2024

(54) METHODS FOR IMPROVING THE MICROBIOME AND ITS SYSTEMIC EFFECT

(71) Applicant: IP SCIENCE LIMITED, Cambridge (GB)

(72) Inventor: Ivan Petyaev, Cambridge (GB)

(73) Assignee: IP SCIENCE LIMITED, Cambridge (GB)

(21) Appl. No.: 18/338,638

(22) Filed: Jun. 21, 2023

Related U.S. Application Data
(63) Continuation-in-part of application No. 17/050,127, filed on Oct. 23, 2020, now Pat. No. 11,717,492.

(30) Foreign Application Priority Data
Apr. 27, 2018 (GB) 1806941.9

(51) Int. Cl. A61K 35/747; A61K 31/01; A61P 1/00; A61P 31/04

(52) U.S. Cl. CPC A61K 35/747; A61K 31/01; A61P 1/00; A61P 31/04

(57) ABSTRACT

The present disclosure relates to administration of carotenoids to provide a prebiotic effect for modification of gut microbiome, thereby increasing counts of Bifidobacterium adolescentis and/or decreasing counts of pathology-associated bacteria from phylum Bacteroidetes, and its particular genus Prevotella, and also genera from other phyla such as Desulfovibrio and Dialister, and resulting in improved systemic effects.

### SEQUENCE LISTING

A sequence listing in electronic (XML file) format is filed with this application and incorporated herein by reference. The name of the ASCII text file is “2023-0932-2.XML”; the file was created on Jul. 20, 2023; the size of the file is 3,615 bytes.

### FIELD OF THE INVENTION

The present disclosure relates to administration of carotenoids to provide a prebiotic effect for improving gut, mouth, skin respiratory and urogenital system microbiome, thereby increasing counts of a genus Bifidobacterium, in particular Bifidobacterium adolescentis and Bifidobacterium longum, and/or decreasing counts of pathology-associated bacteria from phylum Bacteroidetes, and its particular genus Prevotella, and also genera from other phyla such as Desulfovibrio and Dialister, and resulting in improved systemic health beneficial effects.

### BACKGROUND OF THE INVENTION

Significant interest has evolved regarding the gut microbiota, i.e., the entire population of microorganisms that colonizes a particular location, in recent years. The gut microbiota has been associated with a large array of human diseases ranging from luminal diseases, such as inflammatory bowel diseases (IBD) and irritable bowel syndrome (IBS), allergies, metabolic diseases, such obesity and diabetes, to neurodevelopmental illnesses. There is mounting evidence that the gut microbiota has a significant role in maintaining the gut and human health as a whole.

Prebiotics are non-microbial substances that can exert beneficial effects on the human body by helping the body's natural gut microflora to grow and/or by increasing their metabolic activity. The prebiotic concept was first proposed by Gibson and Roberfroid in 1995. The key aspects of a prebiotic are that it is not digestible by the host, and that it leads to health benefits for the individual through a positive influence on native beneficial microbes. The administration or use of prebiotics or probiotics is intended to influence the gut environment, i.e., the microbiota, which is dominated by trillions of commensal microbes, for the benefit of human health. Prebiotics are dietary substances (mostly consisting of non-starch polysaccharides and oligosaccharides). Most prebiotics are used as food ingredients, for example in biscuits, cereals, chocolate, spreads, and dairy products. Commonly known prebiotics are: oligofructose, inulin, galacto-oligosaccharides, lactulose and breast milk oligosacchari des (World Gastroenterology Organisation Global Guidelines, Probiotics and prebiotics, 2017).

Prebiotics are often used in combination with probiotic microorganisms. Probiotics are live microbes that can be formulated into many different types of products, including foods, drugs, and dietary supplements. Species of Lactobacillus and Bifidobacterium are most commonly used as probiotics, but the yeast Saccharomyces cerevisiae and some E. coli and Bacillus species are also been used as probiotics. Lactic acid bacteria (LAB), including species of Lactobacillus, which have been used for preservation of food by fermentation for thousands of years, can serve a dual function by acting as agents of food fermentation and, in addition, potentially imparting health benefits.

Prebiotics and probiotics have been shown to have a number of health benefits. Any composition that combines a probiotic and prebiotic is termed a symbiotic.

Carotenoids are the essential micronutrients, which cannot be synthesized by humans and must be obtained from food. Lycopene, the red pigment of tomatoes, watermelon and some other fruits is one major carotenoid. The current consensus on the broad beneficial effects of lycopene on health is that its powerful antioxidant properties can protect lipoproteins and other lipid structures from their oxidative damage, which typically occurs in a number of pathological conditions.

Although various prebiotic compositions are known in the art, there remains a need for effective prebiotic compositions which support the growth of health-beneficial bacteria. The present invention is aimed at addressing this need.

### SUMMARY OF THE INVENTION

There is emerging evidence that dysbiosis of the gut microbiome on the phylum and genus level can alter representation of bacterial genes and their metabolic pathway, which in turn may contribute to the development of pathogenesis of a number of conditions, including obesity, cardiovascular, neurodegenerative, skin and other diseases. An increase in Bacteroides has been implicated in the development of obesity. Elevated level of representative of other phyla such as genus Desulfovibrio is linked to development of Parkinson's and Alzheimer's diseases, and increase of genus Dialister is responsible for inflammatory gum disease and peridontosis.

Applicants found that continued intervention with carotenoids resulted in a significant decrease in the abundance of Bacteroidetes and genera from other phyla Desulfovibrio and Dialister, and an increase in Actinobacteria, in particular in Bifidobacteria. Bifidobacteria are believed to exert positive health benefits on their host.

Applicants surprisingly found that carotenoids may provide a prebiotic effect on the gut and skin microbiome. Accordingly, some embodiments relate to methods for increasing the population of health-beneficial and decreasing presence of pathology-associated bacteria in the gut and skin, methods for prophylactic and therapeutic treatment of the gut microbiome, compositions for therapeutic treatment of the gut microbiome, as well as carotenoids for use as a prebiotic with systemic health beneficial effect to other organs in the body.

Some embodiments relate to a method for inhibiting growth or decreasing the abundance of Gram-negative anaerobic pathogenic bacteria in the gastro-intestinal tract, mouth, skin, respiratory or urogenital system of a subject in need thereof, the method comprising administering to the subject a therapeutically effective amount of a composition comprising a carotenoid compound, wherein the bacteria is of the genera Desulfovibrio, Prevotella and/or Dialister.

According to some embodiments, the carotenoid compound may be selected from the group consisting of lycopene, lutein, zeaxanthin, meso-zeaxanthin, astaxanthin, α- or β-carotenes, cryptoxanthin, flavoxanthin, neoxanthin, or another tetraterpenoid. Preferably, the carotenoid compound is a lycopene.

According to some embodiments, the method may be for prophylactic treatment of a condition associated with the status of the gastro-intestinal tract, mouth, skin, respiratory or urogenital system health.

According to some embodiments, the condition may be selected from the group consisting of gastrointestinal diseases, inflammatory bowel syndrome, constipation, diarrhoea, colitis, Crohn's disease, appendicitis, ulcerated colitis, functional bowel disorder, irritable bowel syndrome, periodontitis, gingivitis, other mouth pathologies, skin diseases, dermatitis, psoriasis, neurodegenerative diseases, Parkinson's disease, Alzheimer's disease, other forms of dementia, multiple sclerosis, colon cancer, breast cancer, other forms of cancer, urogenital system pathologies, other disorders associated with growth or inflammation associated with or caused by these bacteria or their metabolites, including metabolic syndrome, obesity, cardio- and cerebrovascular diseases, mental pathologies, ageing processes, or any combination thereof.

According to some embodiments, the method may include administering one or more probiotic bacteria. According to some embodiments, the one or more probiotic bacteria may be selected from the group consisting of Lactobacillus plantarum, Lactobacillus acidophilus, Lactobacillus paracasei, Leuconostoc mesenteroides, Lactobacillus bulgaricus, Lactobacillus casei, Lactobacillus salivarius, Acidophilus, Acidophilus Bifidus, Acidophilus Lactobacillus, L. Acidophilus, L. Amylovorus, L. Brevis, L. Casei Immunitas, L. Crispatus, L. Delbrueckii, L. Fermentum, L. Gallinarum, L. Helveticus, L. Johnsonii, L. Johnsonii LC-1, L. Lactis, L. Plantarum, L. Reuteri, L. Rhamnosus, L. Salivarius, L. Sporogenes, Lacto Bacillus, Lactobacille, Lactobacilli, Lactobacilli Bulgaricus, Lactobacilli Plantarum, Lactobacilli Rhamnosus, Lactobacilli Salivarium, Lactobacillus acidophilus, Lactobacillus amylovorus, Lactobacillus brevis, Lactobacillus bulgaricus, Lactobacillus casei, Lactobacillus casei sp. Rhamnosus, Lactobacillus crispatus, Lactobacillus delbrueckii, Lactobacillus delbrueckii ssp. Bulgaricus, Lactobacillus fermentum, Lactobacillus gallinarum, Lactobacillus Gasseri, Lactobacillus GG, Lactobacillus Helveticus, Lactobacillus johnsonii, Lactobacillus Lactis, Lactobacillus reuteri, Lactobacillus Rhamnosus GG, Lactobacillus rhamnosus, Lactobacillus sakei, Lactobacillus sporogenes, Lactobacilo, Lactospores, LC-1, Probiotics, Probiotiques, Pediococcus pentosaceus, Streptococcus thermophilus, Bacillus subtilis, Bacillus coagulans, Enteroccous faecium, Bifidobacterium bifidum, Bifidobacterium lactis, Bifidobacterium Longum, and Bifidobacterium infantis, Bifidobacterium breve, Bifidobacterium animalis, Bifidobacterium longum, or any combination thereof.

According to some embodiments, the composition may be a nutraceutical or liquiceutical, or pharmaceutical composition, or an ingredient of functional food or beverage.

According to some embodiments, the composition comprises at least about 30% w/w, or at least about 50% w/w of the carotenoid.

### BRIEF DESCRIPTION OF THE DRAWINGS

Examples illustrative of embodiments are described below with reference to figures attached hereto. In the figures, identical structures, elements, or parts that appear in more than one figure are generally labeled with the same numeral in all the figures in which they appear. Alternatively, elements or parts that appear in more than one figure may be labeled with different numerals in the different figures in which they appear. Dimensions of components and features shown in the figures are generally chosen for convenience and clarity of presentation and are not necessarily shown in scale. The figures are listed below.

FIG. 1. Changes in the gut microbiome after supplementation with 7 mg and two 30 mg lycopene formulations for one month:
2nd group—7 mg lycopene in cocoa butter.
3rd group—30 mg lycopene in cocoa butter.
4th group—30 mg lycopene sunflower oil.

An increase of Actinobacteria and reduction of Bacteroidetes was detected across the groups, with a proportional increase in relative abundance of Firmicutes for groups 2 and 4.

FIG. 2. Changes in the gut microbiome after continuous ingestion of dark chocolate, its formulation with 7 mg lycopene and control lycopene capsules for one month.

1st group—10 g dark chocolate with 7 mg lycopene.
2nd group—7 mg lycopene in cocoa butter, control in a capsule form. 5th group-10 g of dark chocolate control.

### DETAILED DESCRIPTION OF THE INVENTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Some embodiments relate to the beneficial effect of lycopene on the gut microbiota of humans. Surprisingly, administration of lycopene resulted in a significant prebiotic effect. The positive changes in the gut microbiota profile were accompanied by systemic improvement of different parameters in the body of the participants.

Some embodiments relate to use of a carotenoid compound in promoting growth and/or increasing the abundance of an Actinobacterium, for example Bifidobacterium, in the gastrointestinal tract of a subject.

Some embodiments relate to use of a carotenoid compound to make medical and/or functional food, functional beverage, nutraceutical, or pharmaceutical products for promoting growth and/or increasing the abundance of an Actinobacterium, for example Bifidobacterium, in the gastrointestinal tract of a subject. Each possibility is a separate embodiment.

Some embodiments relate to use of a composition comprising a carotenoid compound to make medical and/or functional food, functional beverage, nutraceutical, or pharmaceutical products for inhibiting growth or decreasing the abundance of Gram-negative anaerobic pathogenic bacteria. Each possibility is a separate embodiment. Some embodiments relate to use of a carotenoid compound for inhibiting growth or decreasing the abundance of Gram-negative anaerobic pathogenic bacteria in the gastro-intestinal tract, mouth, skin, respiratory or urogenital system of a subject. According to some embodiments, the pathogenic bacteria may be of the order Desulfovibrio, Prevotella and/or Dialister. Each possibility is a separate embodiment.

Some embodiments relate to a method for promoting growth and/or increasing the abundance of an Actinobacterium, for example Bifidobacterium, in the gastrointestinal tract of a subject, comprising administration of a carotenoid.

Some embodiments relate to use of a carotenoid compound in decreasing growth and/or decreasing the abundance of pathology-associated members of the phyla Bacteroidetes, and its particular genus Prevotella, and also genera from other phyla such as Desulfovibrio and Dialister in the gastrointestinal tract of a subject.

Some embodiments relate to use of a carotenoid compound to make medical and/or functional food, functional beverage, nutraceutical, or pharmaceutical products for decreasing growth and/or decreasing the abundance of pathology-associated members of the phyla Bacteroidetes, and its particular genus Prevotella, and also genera from other phyla, such as Desulfovibrio and Dialister in the gastrointestinal tract of a subject. Each possibility is a separate embodiment.

Some embodiments relate to a method for inhibiting growth and/or decreasing the abundance of pathology-associated members of the phyla Bacteroidetes, and its particular genus Prevotella, and also genera from other phyla, such as Desulfovibrio and Dialister in the gastrointestinal tract of a subject, comprising administration of a carotenoid.

Some embodiments relate to a method for inhibiting growth or decreasing the abundance of Gram-negative anaerobic pathogenic bacteria in a subject in need thereof, the method comprising administering to the subject a therapeutically effective amount of a composition comprising a carotenoid compound. According to some embodiment, the method is for inhibiting growth or decreasing the abundance of Gram-negative anaerobic pathogenic bacteria in the gastro-intestinal tract, mouth, skin, respiratory or urogenital system of a subject in need thereof, the method comprising administering to the subject a therapeutically effective amount of a composition comprising a carotenoid compound. According to some embodiment, the bacteria is of the genera Desulfovibrio, Prevotella and/or Dialister. Each possibility is a separate embodiment.

According to some embodiments, the subject suffers from a condition selected from the group consisting of gastrointestinal diseases, such as, inflammatory bowel syndrome, constipation, diarrhoea, colitis, Crohn's disease, appendicitis, ulcerated colitis, functional bowel disorder, irritable bowel syndrome, periodontitis, gingivitis or other mouth pathologies, skin deceases diseases, such dermatitis, or psoriasis, neurodegenerative diseases, degasses such Parkinson's disease, Alzheimer's disease, or other forms of dementia, multiple sclerosis, or colon cancer, or breast cancer, or other forms of cancer, or urogenital system pathologies, or other disorders associated with growth or inflammation associated with and/or caused by a member of the phyla Bacteroidetes or their metabolites, such as metabolic syndrome, obesity, cardio- and cerebrovascular diseases, mental pathologies, or ageing processes. Each possibility is a separate embodiment.

Some embodiments relate to a method for the prophylaxis or treatment of a gastrointestinal condition in a subject, comprising administration of a carotenoid.

Some embodiments relate to a method for the prophylaxis or treatment of a condition which can be administration of a carotenoid. In one embodiment, said condition is a result of dysbiosis, that is an imbalanced microbiome.

Some embodiments relate to a method for stimulating beneficial Bifidobacterium microflora in a subject.

Some embodiments relate to a composition comprising a carotenoid and one or more probiotic bacteria.

Some embodiments relate to a composition comprising a carotenoid and optionally one or more probiotic bacteria for use in the treatment of dysbiosis, of a gastrointestinal disease and/or for promoting growth or increasing the abundance of an Actinobacterium, for example Bifidobacterium, in the gastrointestinal tract of a subject.

Some embodiments relate to a composition comprising a carotenoid and optionally one or more probiotic bacteria for use in the treatment of a disease which can be dependent on or associated with the status of the gastrointestinal health.

According to some embodiments, the methods and uses provided herein may provide a systemic health beneficial effect.

Carotenoid compounds are tetraterpenoids which contain long polyene chains.

Carotenoid compounds may include xanthophylls, such as lutein, zeaxanthin, astaxanthin and carotenes, such as beta-carotene, alpha-carotene, zeta-carotene, and lycopene compounds. Each possibility is a separate embodiment.

In one embodiment of the various aspects set out herein, the carotenoid is a xanthophyll. In one embodiment, the xanthophyll is selected from the group consisting of a-cryptoxantin, 13-cryptoxantin, adonirubin, adonixanthin, alloxanthin, amarouciaxanthin A, antheraxanthin, astaxanthin, auroxanthin, caloxanthin, cantaxanthin, capsanthin, capsanthin-5-6-epoxide, capsorubin, crocoxanthin, diadinoxanthin, diatoxanthin, echinenone, fucoxanthin, fucoxanthinol, iso-fucoxanthin, iso-fucoxanthinol, lutein, luteoxanthin, mutatoxanthin, neoxanthin, nostoxanthin, violaxanthin, zeaxanthin, and any combination thereof. Each possibility is a separate embodiment.

In one embodiment, the carotenoid is a carotene. In another embodiment, the carotene is selected from the group consisting of a-carotene, b-carotene, y-carotene, d-carotene, e-carotene, z-carotene, lycopene, neurosporene, phytoene, phytofluene, and any combination thereof. Each possibility is a separate embodiment.

In one embodiment, the carotenes and xantophylles described above may refer to the all-trans forms thereof. In another embodiment, the xantophylles and carotenes for use in the aspects of the present invention may include derivatives containing one or more cis double bonds.

In one embodiment, the carotenoid compound may be a lycopene compound. Lycopene compounds may include lycopene, 1-HO-3′,4′-didehydrolycopene, 3,V—(HO) 2-gamma-carotene, 1, V—(HO) 2-3, 4, 3′, 4′-tetradehydrolycopene, 1,1′-(HO) 2-3, 4-didehydrolycopene. Each possibility is a separate embodiment. In some embodiments, the carotenoid compound is a lycopene compound, such as lycopene. Lycopene is an open-chain unsaturated C40 carotenoid of structure I (Chemical Abstracts Service Registry Number 502-65-8, C₄₀H₅₆).

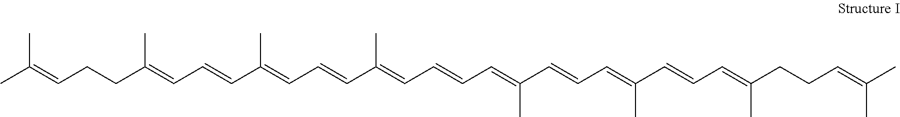 [SMILES: CC(C)=CCC/C(C)=C/C=C/C(C)=C/C=C/C(C)=C/C=C/C=C(C)/C=C/C=C(C)/C=C/C=C(\C)CCC=C(C)C]

Lycopene occurs naturally in plants such as tomatoes, guava rosehip, watermelon and pink grapefruit and any such sources of lycopene may be, for instance, employed in accordance with some embodiments.

According to some embodiments, Lycopene for use as described herein may comprise one or more different isomers. For example, lycopene may include cis-lycopene isomers, trans-lycopene isomers and mixtures of the cis- and trans-isomers. Lycopene may comprise at least about 10%, at least about 20%, at least about 30%, at least about 40%, at least about 50%, at least about 60%, at least about 70%, at least about 80%, at least about 90%, or at least about 95% (Z)-isomers, (all-E)-isomers, or cis-isomers, such as 5-cis- or 9-cis- or 13-cis-isomers. Cis isomers may have improved bioavailability relative to trans isomers. Trans isomers may isomerise into cis forms in vivo, or during storage and processing.

According to some embodiments, carotenoid compounds, such as lycopene, may be natural, i.e., obtained from a natural source, for example, extracted from a carotenoid-rich fruit, vegetable, or other plant, such as a tomato or melon, or from fungi, algae, or bacteria. Each possibility is a separate embodiment. According to some embodiments, the carotenoid compound may be, or comprise, oleoresin, particularly tomato oleoresin.

A range of methods for extracting, concentrating and/or purifying carotenoids from plants are known in the art. According to some embodiments, solvent extraction using ethanol, DMSO, ethyl acetate, hexane, acetone, soya or other vegetable oil, or non-vegetable oils may be employed to extract, concentrate and/or purify carotenoids from plants.

According to some embodiments. carotenoid compounds, such as lycopene, for use as described herein may be synthetic i.e., produced by artificial means, for example, by chemical synthesis. A range of methods for chemical synthesis of lycopene and other carotenoids are known in the art. For example, a three-stage chemical synthesis based on the standard Wittig olefination reaction scheme for carotenoid synthesis may be employed, in which an organic solution of C15 phosphonium methanesulfonate in dichloromethane (DCM) and an organic solution of C10 dialdehyde in toluene may be produced, and the two organic solutions may be gradually combined with sodium methoxide solution and undergo a condensation reaction to form crude lycopene. The crude lycopene may then be purified using routine techniques, for example by adding glacial acetic acid and deionized water to the mixture, stirring vigorously, allowing the aqueous and organic phases to separate, and extracting the organic phase containing DCM and crude lycopene with water. Methanol may be added to the lycopene solution, then heated and cooled to produce a crystalline slurry which may be filtered and washed with methanol. The lycopene crystals may then be recrystallized and dried under heated nitrogen. Synthetic carotenoids, such as lycopene, may also b e available from commercial suppliers (e.g., BASF Corp, NJ USA).

According to some embodiments, synthetic carotenoid compounds, such as lycopene, may comprise an increased proportion of cis isomers relative to natural carotenoid compounds. For example, synthetic lycopene may be up to about 25% 5-cis lycopene, about 1% 9-cis lycopene, about 1% 13-cis lycopene, and about 3% other cis lycopene isomers, whilst lycopene produced by tomatoes may be about 3-5% 5-cis lycopene, about 0-1% 9-cis lycopene, about 1% 13-cis lycopene, and <about 1% other cis lycopene isomers. Since cis-lycopene has increased bioavailability relative to trans-lycopene, synthetic lycopene may be preferred in some embodiments.

According to some embodiments, derivatives of carotenoids as described above may be produced by chemical synthesis analogous to the synthesis described above or by chemical modification of natural carotenoids extracted from plant material.

According to some embodiments of the various aspects set out herein, the Bifidobacterium may be selected from the group including: Bifidobacterium adolescentis, Bifidobacterium catenulatum, Bifidobacterium pseudocatenulatum, Bifidobacterium longum, Bifidobacterium angulatum, Bifidobacterium kashiwanohense, Bifidobacterium dentum and Bifidobacterium stercoris.

According to some embodiments, the subject may have moderate obesity with about 30<BMI<about 35 kg/m².

According to some embodiments, the gastrointestinal condition may be selected from the group including inflammatory bowel syndrome, constipation, diarrhoea, colitis, Crohn's disease, colon cancer, functional bowel disorder, irritable bowel syndrome, periodontitis, gingivitis or other mouth pathologies, etc. Each possibility is a separate embodiment. According to some embodiments, a condition dependent on or associated with gut health can be selected from metabolic syndrome, obesity, cardio- and cerebrovascular disease, aging, neurodegenerative, such Parkinson's disease, Alzheimer's disease or other forms of dementia, multiple sclerosis, skin diseases, such dermatitis or psoriasis, cancer, such as colon or breast cancer or other forms of cancer, urogenital system pathologies, other disorders associated with growth or inflammation associated with or caused by bacteria or their metabolites, such as metabolic syndrome, obesity, cardio- and cerebrovascular diseases, mental pathologies, ageing processes, and any combination thereof. Each possibility is a separate embodiment.

According to some embodiments, the amount of the carotenoid compound effective and/or active in the treatment of a particular disorder and/or condition may depend on the nature of the disorder or condition, and may be determined by standard clinical techniques. Additionally, and/or alternatively, in vitro and/or in vivo assays may optionally be employed to help identify optimal dosage ranges. According to some embodiments, the precise dose to be employed in the compositions may depend on the route of administration, and/or the seriousness of the disease or disorder, and/or may be decided according to the judgment of the practitioner and each patient's circumstances. According to some embodiments, factors, such as age, body weight, sex, diet, time of administration, rate of excretion, condition of the host, drug combinations, reaction sensitivities and severity of the disease may be taken into account.

As used herein, according to some embodiments, the term “effective amount” may relate to an amount of the carotenoid compound, that when administered to a subject, is effective to achieve the desired therapeutic or prophylactic effect under the conditions of administration. As used herein, according to some embodiments, the term “subject” may relate to an animal, for example a mammal, for example a human, dog, cat, or horse, etc. or a non-mammalian species, such as a bird, fish, etc.

According to some embodiments, administration of the carotenoid compound may be co-administration with one or more probiotic bacteria.

Some embodiments may relate to a composition comprising a carotenoid compound and one or more probiotic bacteria.

Some embodiments may relate to a composition comprising a carotenoid compound and optionally one or more probiotic bacteria for use in the treatment of a gastrointestinal disease and/or for promoting growth and/or increasing the abundance of a Bifidobacterium in the gastrointestinal tract of a subject.

According to some embodiments, the amount of the carotenoid compound may be at least about 0.0001% by weight of the composition. According to some embodiments, when intended for oral administration, the amount of the carotenoid compound may be varied to range from about 0.0001% to about 80% by weight of the composition. Preferred oral compositions may comprise from about 0.0004% to about 50% of the carotenoid compound by weight of the composition.

According to some embodiments, some compositions of the present invention may be prepared such that a parenteral dosage unit contains from about 0.0001% to about 2% by weight of the carotenoid compound.

According to some embodiments, for administration by injection, the composition may comprise from about 0.1 mg/kg to about 250 mg/kg of the animal's body weight, preferably, between about 0.1 mg/kg and about 20 mg/kg of the animal's body weight, and more preferably about 1 mg/kg to about 10 mg/kg of the animal's body weight. In one embodiment, the composition may be administered at a dose of about 1 to 30 mg/kg, e.g., about 5 to 25 mg/kg, about 10 to 20 mg/kg, about 1 to 5 mg/kg, or about 3 mg/kg. Each possibility is a separate embodiment. According to some embodiments, the dosing schedule may vary from e.g., once a week to once every 2, 3, or 4 weeks.

According to some embodiments, for oral administration, the amount of carotenoid, for example lycopene, may be between about 1 mg to about 60 mg per day, for example about 5, 7, 10, 20, 30, 40 mg per day. According to some embodiments, administration may be for at least 4 weeks.

According to some embodiments, the amount of carotenoid administered may be dependent on various parameters, such as but not limited to the condition, severity of the condition, age, gender, weight, diet, smoker, non-smoker, blood pressure, comorbidities, co-administered medication, counterindications, dosage form, initial amount of pathogenic bacteria in the gastrointestinal tract of a subject, initial amount of a Bacteroidetes in the gastrointestinal tract of a subject, initial amount of a Actinobacterium in the gastrointestinal tract of a subject, initial amount of a Bifidobacterium in the gastrointestinal tract of a subject, etc., and any combination thereof.

According to some embodiments, the composition may be a pharmaceutical composition of a food supplement composition.

According to some embodiments, the composition may be a nutraceutical composition.

According to some embodiments, the composition may comprise at least about 20% w/w, at least about 30% w/w, at least about 40% w/w, at least about 50% w/w, or at least about 60% w/w of the carotenoid. In one embodiment, the carotenoid compound may be provided as part of a composition which includes an acceptable carrier.

According to some embodiments, the composition may be administered by any convenient route, including but not limited to oral, topical, parenteral, sublingual, rectal, vaginal, ocular, intranasal, pulmonary, intradermal, intrasynovial, epidural, intravitrial, intramuscular, intraperitoneal, intravenous, subcutaneous, intracerebral, transdermal, transmucosal, by inhalation, or topical, particularly to the ears, nose, eyes, or skin or by inhalation. Each possibility is a separate embodiment. In one embodiment, preferably, the compositions may be administered orally.

According to some embodiments, parenteral administration may include, for example, intravenous, intramuscular, intraarterial, intraperitoneal, intrasynovial, epidural, intranasal, rectal, intravesical, intradermal, topical, or subcutaneous administration. Preferably, the compositions may be administered parenterally.

According to some embodiments, the acceptable carrier and/or vehicle may be particulate, so that the compositions are, for example, administered in a tablet, capsule, or compound. According to some embodiments, the acceptable carrier may be liquids, such as water and/or oils. The oils may including those of petroleum, animal, vegetable or synthetic origin, such as peanut oil, soybean oil, mineral oil, sesame oil and the like. Each possibility is a separate embodiment. Preferably, the carrier may be and/or include water. According to some embodiment, saline solutions, aqueous dextrose, and/or glycerol solutions may be employed as liquid carriers, particularly for injectable solutions.

According to some embodiments, the carrier may include saline, gum acacia, gelatin, starch paste, talc, keratin, colloidal silica, urea, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like, and any combination thereof. Each possibility is a separate embodiment.

Additionally, and/or alternatively, auxiliary, stabilizing, thickening, lubricating and/or coloring agents may be used in the composition. Each possibility is a separate embodiment. The compositions may optionally contain minor amounts of wetting agents, emulsifying agents, and/or pH buffering agents.

According to some embodiments, the composition may be in the form of a liquid, e.g., a solution, emulsion, or suspension. The liquid may be useful for delivery of a carotenoid compound by injection, infusion (e.g., IV infusion) or subcutaneously. Each possibility is a separate embodiment. According to some embodiments, in a composition for administration by injection, one or more of a surfactant, preservative, wetting agent, dispersing agent, suspending agent, buffer, stabilizer and isotonic agent may be included. Each possibility is a separate embodiment.

According to some embodiments, when intended for oral administration, the composition may be in a solid or a liquid form, e.g., an elixir, syrup, solution, emulsion, or suspension, where semi-solid, semi-liquid, suspension and gel forms are included within the forms considered herein as either solid or liquid.

According to some embodiments, when intended for oral administration, a composition may comprise one or more of a sweetening agent, preservatives, dye, colorant, flavour enhancer, and any combination thereof. Each possibility is a separate embodiment.

According to some embodiments, as a solid composition for oral administration, the composition may be formulated into a powder, granule, compressed tablet, pill, capsule, chewing gum, wafer or the like form. Each possibility is a separate embodiment. Such a solid composition may contain one or more inert diluents. Additionally, and/or alternatively, one or more of the following may be present in the composition: inert diluents, such as talc, silicone, etc.; binders, such as carboxymethylcellulose, ethyl cellulose, microcrystalline cellulose, or gelatin; excipients, such as starch, lactose or dextrins; disintegrating agents, such as alginic acid, sodium alginate, corn starch and the like; lubricants, such as magnesium stearate; glidants, such as colloidal silicon dioxide; sweetening agents, such as sucrose or saccharin; a flavoring agent, such as peppermint, methyl salicylate or orange flavoring; a coloring agent; and any combination thereof. Each possibility is a separate embodiment.

According to some embodiments, when the composition is in the form of a capsule (e.g., a gelatin capsule), it may contain, in addition to materials of the above type, a liquid carrier, such as polyethylene glycol, cyclodextrin and/or a fatty oil.

According to some embodiments, compositions may be formulated to release the carotenoid compound substantially immediately upon administration or at any predetermined time or time period after administration. Each possibility is a separate embodiment. In one example, controlled release may be obtained by appropriate selection of various formulation parameters and excipients, including, e.g., various types of controlled release compositions and coatings. Thus, the carotenoid compound may be formulated with appropriate excipients into a pharmaceutical composition that, upon administration, may release the therapeutic carotenoid in a controlled manner. Examples include single or multiple unit tablet or capsule compositions, oil solutions, suspensions, emulsions, microcapsules, microspheres, molecular complexes, nanoparticles, patches, and liposomes. Each possibility is a separate embodiment.

According to some embodiments, compositions may take the form of one or more dosage units.

In one embodiment, administration of the carotenoid compound may be as part of a combination therapy. The carotenoid compound may be co-administered with the other therapy simultaneously, sequentially, or given at another time. Each possibility is a separate embodiment.

In another embodiment, the subject may have previously received another therapy.

According to some embodiments, the composition may include a carotenoid compound and one or more probiotic bacteria. The composition may be a pharmaceutical composition according to the embodiments above. In one embodiment, the carotenoid compound is lycopene.

In one embodiment, the one or more probiotic bacteria may be selected from the group consisting of: Lactobacillus plantarum, Lactobacillus acidophilus, Lactobacillus paracasei, Leuconostoc mesenteroides, Lactobacillus bulgaricus, Lactobacillus casei, Lactobacillus salivarius, Acidophilus, Acidophilus Bifidus, Acidophilus Lactobacillus, L. Acidophilus, L. Amylovorus, L. Brevis, L. Casei lmmunitas, L. Crispatus, L. Delbrueckii, L. Fermentum, L. Gallinarum, L. Helveticus, L. Johnsonii, L. Johnsonii LC-1, L. Lactis, L. Plantarum, L. Reuteri, L. Rhamnosus, L. Salivarius, L. Sporogenes, Lacto Bacillus, Lactobacille, Lactobacilli, Lactobacilli Bulgaricus, Lactobacilli Plantarum, Lactobacilli Rhamnosus, Lactobacilli Salivarium, Lactobacillus acidophilus, Lactobacillus amylovorus, Lactobacillus brevis, Lactobacillus bulgaricus, Lactobacillus casei, Lactobacillus casei sp. rhamnosus, Lactobacillus crispatus, Lactobacillus delbrueckii, Lactobacillus delbrueckii ssp. bulgaricus, Lactobacillus fermentum, Lactobacillus gallinarum, Lactobacillus Gasseri, Lactobacillus GG, Lactobacillus Helveticus, Lactobacillus johnsonii, Lactobacillus Lactis, Lactobacillus reuteri, Lactobacillus Rhamnosus GG, Lactobacillus rhamnosus, Lactobacillus sakei, Lactobacillus sporogenes, Lactobacilo, Lactospores, LC-1, Probiotics, Probiotiques, Pediococcus pentosaceus, Streptococcus thermophilus, Bacillus subtilis, Bacillus coagulans, Enteroccous faecium, Bifidobacterium bifidum, Bifidobacterium lactis, Bifidobacterium longum, and Bifidobacterium infantis, Bifidobacterium breve, Bifidobacterium animalis or Bifidobacterium longum, and any combination thereof. Each possibility is a separate embodiment.

Some embodiments may relate to a kit comprising a carotenoid compound and one or more probiotic bacteria. Some embodiments may relate to a kit comprising a carotenoid compound, and optionally one or more probiotic bacteria, for use in the treatment of a gastrointestinal disease or a disease associated with the gastrointestinal tract and/or for promoting growth and/or increasing the abundance of an Actinobacterium, for example Bifidobacterium, in the gastrointestinal tract of a subject. Each possibility is a separate embodiment.

Unless otherwise defined herein, scientific and technical terms used in connection with the present disclosure shall have the meaning that are commonly understood by those of ordinary skill in the art. While the foregoing disclosure provides a general description of the subject matter encompassed within the scope of the present invention, including methods, as well as the best mode thereof, of making and using invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

All documents mentioned in this specification are incorporated herein by reference in their entirety.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the term “about” may be used to specify a value of a quantity or parameter (e.g., the length of an element) to within a continuous range of values in the neighbourhood of (and including) a given (stated) value. According to some embodiments, “about” may specify the value of a parameter to be between 80% and 120% of the given value.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

As used herein, the term “and/or” is to be taken as specific disclosure of each of the two specified features or components with or without the other. For example “A and/or B” is to be taken as specific disclosure of each of (i) A, (ii) B and (iii) A and B, just as if each is set out individually herein. Unless context dictates otherwise, the descriptions and definitions of the features set out above are not limited to any particular aspect or embodiment of the invention and apply equally to all aspects and embodiments which are described.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although steps of methods according to some embodiments may be described in a specific sequence, methods of the disclosure may include some or all of the described steps carried out in a different order. A method of the disclosure may include a few of the steps described or all of the steps described. No particular step in a disclosed method is to be considered an essential step of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purposes and should not be regarded as limiting. Citation or identification of any reference in this application shall not be construed as an admission that such reference is available as prior art to the disclosure. Section headings are used herein to ease understanding of the specification and should not be construed as necessarily limiting.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

The following examples are presented to provide a more complete understanding of the invention. The specific techniques, conditions, materials, proportions and reported data set forth to illustrate the principles of the invention are exemplary and should not be construed as limiting the scope of the invention.

The invention is further described in the non-limiting examples.

### Examples

Materials and Methods

Study Design. The total number of volunteers recruited to take part in the study was 30 (15 male and 15 female subjects) Caucasians 40-67 years old. They were randomised and divided into five groups of equal size. Group I received a daily dose of 10 g dark chocolate with 7 mg lycopene by a proprietary composition guaranteeing its maximum embedment into the lipid part of the chocolate, L-Tug, and on another optimal lycopene coating of chocolate crystals and formation of coco-lycosomes, DCL. Group II received daily one capsule of 7 mg GA Lycopene formulated with medium saturated fatty acids, GAL-SMFA. Group III received one capsule daily of 30 mg GAL-SMFA. Group IV received one capsule daily 30 mg of GA lycopene formulated with polyunsaturated fatty acids, GAL-PUFA. Group V received 10 mg of the control dark chocolate daily.

Three GAL (GAL=formulated lycopene) groups received blinded lycopene capsules, as two other groups received blinded DC (dark chocolate) products.

Inclusion Criteria were:
ability to sign an informed consent,
light-to-moderate smokers (<10 cigarettes daily), moderately obese with BMI between 30 and 35 kg/m2, with elevated serum markers of inflammatory oxidative damage, IOD>40 pM/mL and oxidative stress, LDL-Px, ELISA c 103>200,
no participation in other dietary trials during the last 3 months before enrolment and duration of willingness and ability to comply with the study protocol for the duration of the study.

Exclusion criteria were:
unwillingness to sign informed consent,
unable to comply with the protocol for the duration of the study,
history of myocardial infarction in the 3 months preceding the study, ejection fraction (EF)<45%, significant medical condition that would impact safety considerations (e.g., significantly elevated LFT, hepatitis, severe dermatitis, uncontrolled diabetes, cancer, severe GI disease, fibromyalgia, renal failure, recent CVA (cerebrovascular accident), pancreatitis, respiratory diseases, epilepsy, etc.), compulsive alcohol abuse (>10 drinks weekly), or regular exposure to other substances of abuse, participation in other nutritional or pharmaceutical studies, resting heart rate of >100 beats per minute or <50 beats per minute, positive test for tuberculosis, HIV, or hepatitis B, unable to tolerate phlebotomy, special diets in the 4 weeks prior to the study (e.g., liquid, protein, raw food diet), tomato intolerance.

Products. All products for the trial were developed and made by Lycotec Ltd. (Cambridge, United Kingdom). The product was especially designed to improve lycopene bioavailability in middle-aged persons, 50 years old or above, or in those who have such conditions as metabolic syndrome, fatty liver, etc. It contained phosphatidylcholine, which serves as principle scaffolding element for incorporation of lycopene during lipoprotein intracellular re-assembly, the process that is essential for lycopene transportation but impaired in the above individuals.

There were two formulations of GAL, for two different nutraceutical applications, were applied in this study. The first one was with a blend of SMFA to facilitate formation of small-medium chylomicrons, which would be transported by the portal vein for liver targeting delivery of lycopene. The second one was a blend with PUFA to facilitate formation of larger chylomicrons, which would be transported by the thoracic duct for the systemic blood circulation bypassing the liver. All GAL products were made in gelatin capsule.

For the control DC and DCL Green & Black's 70% dark chocolate was used. It was made from Trinitario cocoa beans and contained: 42% fat, of which saturates were 25%; carbohydrates 36.5%, of which sugars were 28.5%; fibre 10%, protein 9.1%, salt 0.13%. Each 10 g bar contained 1.5 mg of catechins, 6.6 mg of epicatechins, 1.9 mg of dimer-B2, 7.5 mg of caffeine, 75 mg of theobromine, 75 μg of phenylethylamine, 55 μg of serotonin, <0.1 μg of resveratrol. Both capsule and chocolate products were advised to be taken once a day after the main meal.

The period of administration was 1 month.

Methods.

BMI, Pulse Rate, and BP.

Measurements of body mass index, BMI, body mass of the patients and their height were carried out in the morning and BMI was calculated in kg/m2. Pulse rate, systolic and diastolic blood pressure, SBP and DBP, were recorded three times on the left arm of the seated patient after 15 min of rest. The time between measurements was greater than 2 minutes. The mean result for each parameter was calculated. All body and vascular parameters were recorded in the morning between 8 and 10 am.

Tissue Oxygenation. Thenar eminence and forearm muscles of the patients were used as a tissue target for the assessment of oxygen saturation, StO2, or combined level of oxygenated haemoglobin and myoglobin. StO2 was assessed by continuous wavelength near-infrared spectroscopy, NIRS, with wide-gap second-derivative (In Spectra, Hutchinson Technology, MN, USA). The measurements were taken at different time points. The recording was initiated after 15 min of rest in a supine position before occlusion of the brachia! artery. It was then continued during stagnant ischemia induced by rapidly inflating the cuff to 50 mm Hg above systolic BP. The ischemia lasted for 3 min, and the recording period lasted for another 5 min after that until StO2 was stabilized. The area under the hyperaemic curve, AUC, of the recorded signal for the settling time in the post-occlusion period was then calculated as described earlier in % 02/minute [Castes F. et al, 1999, Marseglia L. et al, 2105].

Samples Collection. Blood was collected by phlebotomy in the morning, in the hospital, from the arm veins of patients following night fast. The serum was separated from the rest of the clotted mass by centrifugation, aliquots were then stored in code-labeled tubes for blinded analysis and stored at −80° C. until use.

For sample collection from the surface of the facial skin and samples of the cerumen all study participants were requested to avoid facial and ear hygienic manipulations for 24 hours before sampling, which was carried out in the morning in parallel with blood sample collection.

Briefly, samples were collected using polyester swabs from the surface of the facial skin (the sides of the nose). During the procedure two samples were taken (one swab per side). Each collected sample was placed on the surface of a microscope slide. A second microscope slide was pressed against the surface of the first one. This procedure provided a pair of identical smears. All slides with collected samples were coded to provide sample anonymity for blinded analysis and stored at −20° C. until further analysis.

The stool samples were collected either in the morning or night before the day of the visit to the hospital. Volunteers undertook this collection themselves, in the convenience of their own homes. For this purpose, participants used a special kit and sample containers which were provided by the trial team. The collected samples were labeled and stored at −80° C. until their analysis.

Gut Microbiome analysis.

DNA Extraction

One ml of each fermentation endpoint (at 24 h) was pelleted via centrifugation at 13.000 g for 10 min and gDNA was extracted from the pellet using the Power Soil Kit protocol (MoBio Laboratories). The FastPrep bead-beating step was performed in 3 cycles of 15 s each at a speed of 6.5 M/s in a FastPrep-24™ Homogenizer (MP). DNA quantity and quality were measured using a NanoDrop 1000 (Thermo Scientific).

16S rRNA Gene Library Preparation

The fecal microbiota composition of in vitro fermentation samples were determined using tag-encoded 16S rRNA gene MiSeq-based (Illumina, CA, USA) high throughput sequencing. The V3 region of the 16S rRNA gene was amplified using primers compatible with the Nextera Index Kit (Illumina) NXt_338_F:5′-TCGT CGG CAG CGT CAG AT GTGTAT AAG AG AC AG ACWCCT ACGG G WGG CAGCAG-3′ (SEQ ID NO. 1) and NXt_518_R: 5′-GTCTCGTGGGCTCGGAGATGTGTATAAGAGACAGATTACCGCGGCTGCTGG-3′ (SEQ ID NO. 2) [Ovreas et al. 1997]. The PCR reactions and library preparation were conducted as described in [Kristensen et al. 2016].

High Throughput Sequencing and Data Treatment

The raw dataset containing pair-ended reads with corresponding quality scores were merged and trimmed using fastq_mergepairs and fastq filter_scripts implemented in the UPARSE pipeline. The minimum overlap length was set to 10 base pairs (bp). The minimum length of merged reads was 150 bp, the maximum expected error E was 2.0, and the first truncating position with quality score was N:54. Purging the dataset from chimeric reads and constructing de nova Operational Taxonomic Units (OTU) were conducted using the UPARSE pipeline [Edgar 2013]. The Green Genes (13.8) 16S rRNA gene collection was used as a reference database [McDonald et al. 2012]. Quantitative Insight into Microbial Ecology (QIIME) opensource software [Caporaso et al. 2010] (1.7.0 and 1.8.0) was used for the subsequent analysis steps. Principal coordinate analysis (PCoA) plots were generated with the Jackknifed Beta Diversity workflow based on 10 UniFrac distance metrics calculated using 10 subsampled OTU tables. The number of sequences taken for each jackknife subset was set to 90% of the sequence number within the most indigent sample, hence xx000 reads per sample for the inulin and lactulose 16S rRNA library experiments and 87000 reads/sample for the HMO 16S rRNA library/experiments. Analysis of similarities (ANOSIM) was used to evaluate group differences using weighted and unweighted (Lozupone & Knight 2005) UniFrac distance metrics that were generated based on rarefied (xx000 reads/sample) OTU tables. The relative distribution of the genera registered was calculated for unified and summarized in genus level OTU tables. Alpha diversity measures expressed as observed species values (sequence similarity 97%) were computed for rarefied OTU tables (xx000 reads/sample) using the alpha rarefaction workflow. Differences in alpha diversity were determined using at-test-based approach employing the non-parametric (Monte Carlo) method (999 permutations) implemented in the compare alpha diversity workflow. The ANOVA determined significance of quantitative (relative abundance) association of OTUs with given categories, p values were False Discovery Rate (FDR) corrected. These were calculated based on 1000 subsampled OTU-tables rarefied to an equal number of reads (xx000 reads/sample).

Biochemistry. Glucose, total cholesterol, triglycerides, high density cholesterol, low density cholesterol, C-reactive protein were determined using commercially available analytical kits according to the manufacturers' instructions (ByoSystems, R&D Systems).

Lycopene Quantitative Analysis. The lycopene concentration in all serum samples was measured in duplicate by high-performance liquid chromatography with modifications. Briefly, 400 μI of serum was mixed with 400 μI of ethanol and was extracted twice with 2 ml hexane. The combined hexane layers were evaporated to dryness in a vacuum (Scan Speed 32 centrifuge) and the residue reconstituted to a volume of 100 μI in sample solution (absolute ethanol-methylene chloride, 5:1, v/v). The specimens were centrifuged again (15 minutes at 10,000 g) and clear supernatant was transferred to HPLC vials. Five microliters of the extract were injected into an Acquity HSS T3 75×2.1 mm 1.8 μm column (Waters, USA) preceded by a Acquity HSS T3 1.8 μm VanGuard precolumn (Waters, USA) and eluted isocratically at 45° C. with the mobile phase (acetonitrile-0.08% phosphoric acid solution-tert-Butyl methyl ether, 70:5:25, v/v/v) at a flow rate of 0.5 ml/min. The lycopene peak was detected by a Photodiode Array Detector (Waters, USA) at 474 nm. The peak area was measured using Empower 3 software (Waters, MA). The lycopene concentration in serum samples was calculated by reference to an analytical standard (lycopene from tomato, L9879, Sigma, USA).

Inflammatory Oxidative Damage (IOD). Serum samples were incubated overnight in 0.05 M PBS acetate buffer (pH 5.6) to imitate the type of oxidative damage which occurs during the release of lysosomes following neutrophil degranulation. The following morning the reaction was stopped using trichloroacetic acid. The concentration of the end products such as malonic dialdehyde (MDA), and other possible thiobarbituric acid reactive substances (TBARS), was then measured by colorimetry using reagents and kits from Cayman Chemical (MC, USA).

LDL-Px and Lipoprotein 02. Activity of serum LDL peroxidase proteins, which include lgG with superoxide dismutase activity, was measured as described previously [Petyaev]. Plasma oxygen carried by blood lipids/lipoproteins was measured by catalymetry.

Statistics. For the assessment of normally distributed parameters the Shapiro-Wilk method was used. Student's t-test was then applied for both paired and unpaired samples. In cases where parameters were not normally distributed the Mann-Whitney test and Kruskal-Wallis test were used. ANOVA and ANCOVA were used with post hoc analysis (Statistica 9 suite, StatSoft; Inc.). Statistical significance between two-tailed parameters was considered to be P<0.05.

Results

General Characteristics of the Study Population

Baseline characteristics of the participants are presented in Table 1. Apart of their increased BMI all other measured parameters, from cardiovascular to blood biochemistry, were within the norm.

TABLE 1
BASELINE CHARACTERISTICS OF THE ENROLLED VOLUNTEERS
(Mean +/− SD)

| | Groups | | | | |
|---|---|---|---|---|---|
| | I | II | III | IV | V |
| Number of Patients | 6 | 6 | 6 | 6 | 6 |
| Males | 3 | 2 | 4 | 3 | 3 |
| Females | 3 | 4 | 2 | 3 | 3 |
| Age | 61.8 ± 5.9 | 56.2 ± 5.9 | 56.1 ± 5.8 | 52.1 ± 5.1 | 63.2 ± 6.1 |
| Light/Moderate Smokers | 1 | 1 | 1 | 1 | 1 |
| Body Mass Index in | 32.1 ± 2.4 | 32.7 ± 3.3 | 33.8 ± 3.5 | 31.1 ± 3.2 | 31.8 ± 2.9 |
| kg/m² | | | | | |
| Fasting Glucose | 6.1 ± 0.42 | 6.0 ± 0.45 | 5.7 ± 0.49 | 5.4 ± 0.43 | 5.5 ± 0.56 |
| mmol/dL | | | | | |
| Total Cholesterol mg/dl | 185 ± 14.3 | 181 ± 15.2 | 175 ± 14.7 | 187 ± 16.2 | 180 ± 13.9 |
| Triglycerides mg/di | 135 ± 14.9 | 136 ± 13.8 | 136 ± 13.8 | 127 ± 13.1 | 122 ± 13.5 |
| LDL mg/dL | 144 ± 11.8 | 143 ± 12.7 | 121 ± 12.2 | 137 ± 13.6 | 131 ± 12.1 |
| HDL mg/dL | 41.9 ± 3.2 | 46.5 ± 4.4 | 51.2 ± 4.7 | 49.8 ± 4.4 | 44.0 ± 4.4 |
| Pulse rate per min | 66.7 ± 4.2 | 67.7 ± 3.5 | 65.2 ± 3.4 | 70.5 ± 3.9 | 66.6 ± 5.1 |
| Blood Pressure | | | | | |
| Systolic | 112 ± 5.5 | 123 ± 7.4 | 117 ± 6.9 | 124 ± 8.5 | 118 ± 6.7 |
| Diastolic | 77.6 ± 4.4 | 78.7 ± 5.0 | 77.6 ± 4.4 | 76.7 ± 4.6 | 79 ± 5.6 |

Gut Microbiome

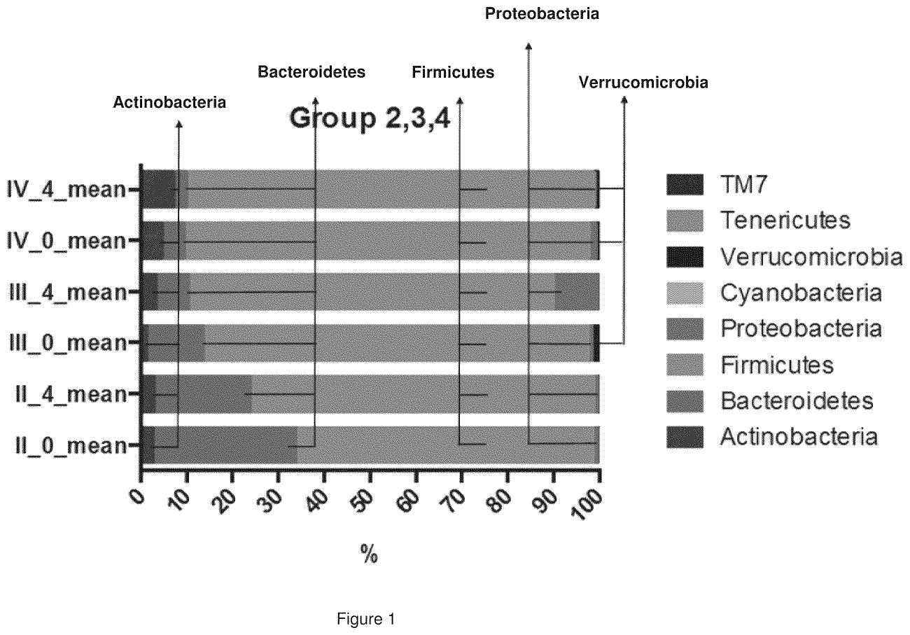

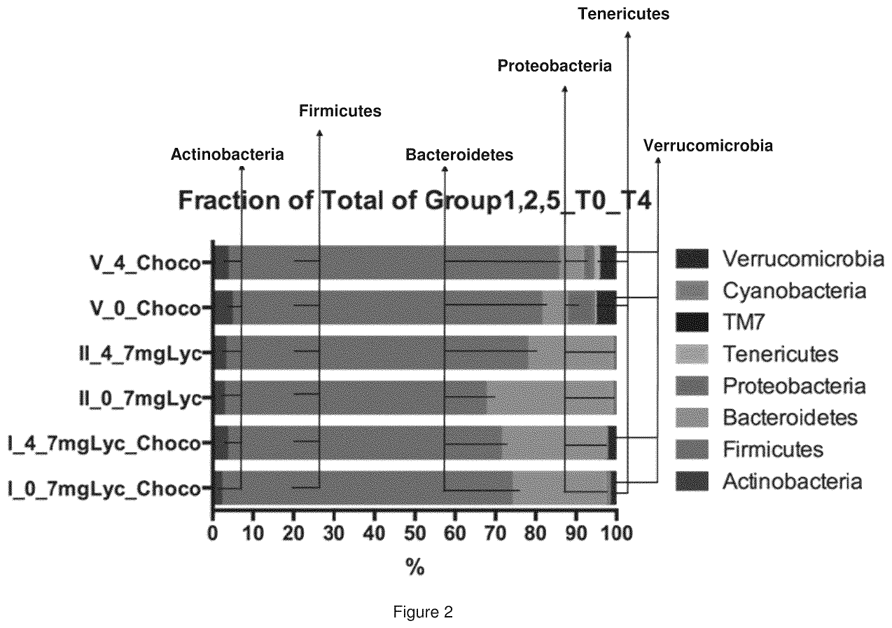

Results of changes in the gut microbiome after one month of supplementation with 20 mg of formulated lycopene are presented in FIG. 1 and FIG. 2.

After 4 weeks of supplementation with GA lycopene, a shift in the gut microbial communities of the intervention participants was detected. The relative abundance of Phyla level changed with a trend to increased relative abundance in Actinobacteria in all intervention groups, Group IV 4.5%-7.12% (p=0.51), Group III with 1.12% 3.22% p=0.04 (FDR corr), Group II, 2.52% 2.85%, p=0.8).

Members of the Phyla Bacteroidetes decreased in the relative abundance in all groups even though not statistically significant, Group IV 4.92%-2.72%, p=0.52; Group III 12.4% to 7.2%, p=0.8 FDR, Group2 31.26% to 21.05%, p=0.43.

Proteobacteria decreased in group IV (Group IV 1.7%-0.15%, p=0.28), whereas they increased in group III 0.9 to 10%, FDR p=0.5) in Group II the relative abundance of Proteobacteria remained the same with 0.95% and 0.92%, p=0.95.

The Phyla composition of the Gut Microbiota from intervention groups is shown in FIG. 1.

An increased dose of Lycopene 30 mg Group III and IV vs Group II, 7 mg, was also reflected in an increased relative abundance of Actinobacteria (+2.6 Group IV, Group III+2.1%, Group II+0.33%).

Relative abundances of Actinobacteria at week 0 and week 4 at species level OTU are shown in Table 2.

When looking at the OTUs representative of the Bacteroidetes phyla it becomes clear that several intervention groups (Group II, III, IV) decreased, but the effect was OTU specific as some members of the Bacteroidetes increased.

The OTU belonging to the family of Prevotellaceae, as well as the OTU Prevotella stercorea, Prevotella, Bacteroides caccae, Prevotella copri, Bacteroidetes—Bacteroides ovatus, an OUT of Paraprevotella decreased (Table 2).

DC Group V dark chocolate decreased the relative abundance of Actinobacteria 4.4-3.4%, p=0.7), Bacteroidetes did not change in relative abundance 6.4%-6.3% (p=0.9), Proteobacteria 6.6-2.4%, p=0.5). DC and Lycopene Group I: Actinobacteria also increased (1.89% to 3.3%, p=0.2) after 4 weeks of intervention) Bacteroidetes on the other hand increased slightly from 23.4-25.8%, p=0.8), Firmicutes decreased (71.7-67.8%, p=0.8), Proteobacteria decreased from 0.49 to 0.24%, p=0.5 (Table 3, Figure. 2).

When looking at Actinobacteria and the changes on the species OTU level, an increase of Bifidobacterium longum and Bifidobacterium adolescentis were observed and another Bifidobacterium species were observed across all GAL intervention.

On the Species OTU level Group I displayed a significant decrease in the OTU Bacteroides unassigned (0.075 to 0.018, p=0.3), Bifidobacterium adolescentis increased 10-fold 0.006-0.06%, p=0.1.

Looking at the members of the phyla Firmicutes, an increase in a Streptococcus OTU was detected across all intervention groups.

The main phyla abundance of which benefited from the lycopene ingestion, whether it was taken in a capsule form or in a chocolate matrix, was Actinobacteria, and in particular Bifidobacteria adolescentis which has probiotic beneficial health effects.

TABLE 2
Average relative species compositions of gut microbial communities for the
intervention groups 7 mg GAL-MSFA, 30 mg GAL-MSFA, 30 GAL-PUFA at different doses
at thebeginning and at the end of the intervention study

| | | | | | | 30 mg | | 30 mg | |
|---|---|---|---|---|---|---|---|---|---|
| | | | | 7 mg | GAL MSFA | III_ | GAL MSFA | IV_ | GAL PUFA |
| Phyla | Family | Genera | Species | II_Day_0 | II_Week_4 | Day_0 | III_Week_4 | Day_0 | IV_Week_4 |
| Bacteroidetes | Porphyromonadaceae | Pamibacteroides | [symbol] | 0.36 | 0.09 | 0.04 | 0.04 | 0.04 | 0.05 |
| Bacteroidetes | S24-7 | | | 0.73 | 1.72 | 0.15 | 0.48 | 0.07 | 0.2 |
| Bacteroidetes | Porphyromonadaceae | Parabacteroides | | 0.33 | 0.1 | 0.68 | 0.09 | 0.05 | 0.24 |
| Bacteroidetes | [ [symbol] ] | Paraprevotella | | 0.11 | 0.05 | 0.26 | 0.03 | 0.01 | 0 |
| Bacteroidetes | Bacteroidaceae | Bacteroides | | 8.59 | 2.58 | 0.91 | 3.14 | 0.74 | 0.95 |
| Bacteroidetes | Bacteroidaceae | Bacteroides | ovatus | 0.08 | 0.04 | 0.03 | 0.02 | 0.01 | 0 |
| Bacteroidetes | | | | 0.77 | 1.14 | 0.31 | 0 | 0.08 | 0.02 |
| Bacteroidetes | Prevotellaceae | Prevotella | copri | 13.52 | 12.2 | 8.52 | 0.38 | 0.8 | 0.13 |
| Bacteroidetes | Bacteroidaceae | Bacteroides | caceae | 0.09 | 0.01 | 0.02 | 0.01 | 0 | 0 |
| Bacteroidetes | Bacteroidaceae | Bacteroides | Other | 0.15 | 0.04 | 0.04 | 0.1 | 0.09 | 0.26 |
| Bacteroidetes | Prevotellaceae | Prevotella | | 0.26 | 0.19 | 0.38 | 0 | 0.2 | 0 |
| Bacteroidetes | Prevotellaceae | Prevotella | stercorea | 1.24 | 0.52 | 0.15 | 0 | 0.11 | 0.03 |
| Bacteroidetes | Rikenellaceae | | | 2.18 | 0.57 | 0.2 | 1.33 | 0.34 | 0.39 |
| Bacteroidetes | Bacteroidaceae | Bacteroides | [symbol] | 0.49 | 0.99 | 0.15 | 1.22 | 2 | 0.01 |
| Bacteroidetes | [Odoribacteraceae] | Butyricimonas | | 0.08 | 0.03 | 0.03 | 0.04 | 0.02 | 0.01 |
| Bacteroidetes | [Paraprevotellaceae] | [Prevotella] | | 1.16 | 0.5 | 0.21 | 0 | 0.04 | 0.02 |
| Bacteroidetes | [Barnesiellaceae] | | | 0.71 | 0.07 | 0.02 | 0.1 | 0.05 | 0.06 |
| Bacteroidetes | Bacteroidaceae | Bacteroides | uniformis | 0.24 | 0.03 | 0.08 | 0.11 | 0.03 | 0.34 |
| Actinobacteria | Bifidobacteriaceae | Bifidobacterium | longum | 0.09 | 0.11 | 0.04 | 0.16 | 0.11 | 0.16 |
| Actinobacteria | Bifidobacteriaceae | Bifidobacterium | | 0.15 | 0.54 | 0.03 | 0.5 | 0.73 | 2.31 |
| Actinobacteria | Bifidobacteriaceae | Bifidobacterium | adolescentis | 0.33 | 1.09 | 0.05 | 0.24 | 0.69 | 2.93 |
| Actinobacteria | Actinomycetaceae | Actinomyces | | 0.02 | 0.01 | 0.04 | 0.35 | 0.04 | 0.02 |
| Actinobacteria | Coriobacteriaceae | Other | Other | 0 | 0 | 0.02 | 0.03 | 0.01 | 0.01 |
| Actinobacteria | Coriobacteriaceae | Eggerthella | lenta | 0.01 | 0 | 0.01 | 0.02 | 0.09 | 0.07 |
| Actinobacteria | Coriobacteriaceae | Atopobium | | 0.01 | 0.01 | 0.01 | 0.09 | 0.02 | 0.02 |
| Actinohactaria | Coriobacteriaceae | Collinsella | aerofaciens | 0.96 | 0.58 | 0.59 | 1.2 | 1.57 | 0.94 |
| Actinobacteria | Coriobacteriaceae | | | 0.83 | 0.41 | 0.21 | 0.27 | 0.91 | 0.52 |
| Actinobacteria | Coriobacteriaceae | Adlercreutzia | | 0.02 | 0.01 | 0.06 | 0.1 | 0.09 | 0.07 |
| Actinobacteria | Coriobacteriaceae | Slakia | | 0.09 | 0.09 | 0.07 | 0.1 | 0.23 | 0.04 |
| Firmicutes | Veillonellaceae | Dialister | | 0.3 | 0.82 | 1.54 | 0.09 | 1.2 | 7.23 |
| Firmicutes | Lachnospiraceae | Anaerostipes | | 0.04 | 0.02 | 0.02 | 0.04 | 0.03 | 0.11 |
| Firmicutes | Clostridiaceae | Caloramator | | 0.01 | 0 | 0.01 | 0.04 | 0.1 | 0 |
| Firmicutes | Streptococcaceae | Streptococcus | | 0.13 | 0.21 | 0.8 | 8.14 | 0.19 | 2.26 |
| Firmicutes | Clostridiaceae | | | 0.95 | 0.75 | 0.82 | 4.8 | 5.63 | 0.94 |
| Firmicutes | Lachnospiraceae | Roseburri | | 0.08 | 0.14 | 0.04 | 0.03 | 0.09 | 0.05 |
| Firmicutes | | | | 6.99 | 6.64 | 16.32 | 10.2 | 8.45 | 5.98 |
| Firmicutes | Clostridiaceae | Other | Other | 0.03 | 0.01 | 0.03 | 0.22 | 0.53 | 0.04 |
| Firmicutes | Lachnospiraceae | [Ruminococcus] | Other | 0.01 | 0.01 | 0.02 | 0 | 0 | 0.01 |
| Firmicutes | Lachnospiraceae | [Ruminococcus] | [symbol] | 0.04 | 0.04 | 0.05 | 0.06 | 0.13 | 0.06 |
| Firmicutes | Lachnospiraceae | Blautia | Other | 0.86 | 0.68 | 1.09 | 1.02 | 1.72 | 2.23 |
| Firmicutes | Lachnospiraceae | Blautia | | 3.08 | 2.23 | 3.78 | 4.24 | 7.24 | 9.07 |
| Firmicutes | Lachnospiraceae | Ceprecoccus | Other | 0.13 | 0.22 | 0.36 | 0.15 | 0.15 | 0.15 |
| Firmicutes | Ruminococcaceae | Faecalibacterium | [symbol] | 1.39 | 2.42 | 1.04 | 1 | 0.98 | 1.13 |
| Firmicutes | Veillonellaceae | [symbol] | | 0.39 | 0.17 | 0.04 | 0.02 | 3.69 | 0.01 |
| Firmicutes | Ruminococcaceae | | | 20.99 | 30.92 | 25.11 | 15.39 | 20.02 | 23.35 |
| Firmicutes | Other | Other | Other | 1.31 | 0.77 | 1.59 | 6.12 | 4.79 | 1.8 |
| Firmicutes | Streptococcaceae | Streptococcus | [symbol] | 0.01 | 0 | 0.02 | 0.08 | 0 | 0.11 |
| Firmicutes | Lachnospiraceae | [Ruminococcus] | | 0.2 | 0.31 | 0.17 | 0.19 | 0.26 | 0.17 |
| Firmicutes | [symbol] | | | 0.08 | 0.04 | 0.02 | 0.09 | 0.2 | 0.05 |
| Firmicutes | Christensenellaceae | | | 0.23 | 0.28 | 0.18 | 0.04 | 0.93 | 0.24 |
| Firmicutes | Lachnospiraceae | Shuttleworthia | | 0.12 | 0.1 | 0.13 | 0.15 | 0.18 | 0.21 |
| Firmicutes | Clostridlaceae | Clostridium | | 0.32 | 0.45 | 0.3 | 0.42 | 0.3 | 0.27 |
| Firmicutes | Lachnospiraceae | | | 7.01 | 8.06 | 8.82 | 7.27 | 9.5 | 8.18 |
| Firmicutes | Ruminococcaceae | Oscillospira | | 1.71 | 1.44 | 1.69 | 2.56 | 1.98 | 1.66 |
| Firmicutes | [symbol] | ( [symbol] ) | [symbol] | 0.53 | 0.27 | 0.38 | 1.03 | 0.78 | 0.09 |
| Firmicutes | Lachnospiraceae | [symbol] | | 0.25 | 0.11 | 0.23 | 0.04 | 0.23 | 0.21 |
| Firmicutes | [Mogibacteriaceae] | | | 0.17 | 0.11 | 0.06 | 0.08 | 0.29 | 0.11 |
| Firmicutes | Lachnospiraceae | Other | Other | 4.85 | 5.88 | 5.46 | 4.67 | 7.47 | 6.19 |
| Firmicutes | Lachnospiraceae | Coprococcus | | 1.78 | 2.07 | 2.2 | 2.56 | 3.41 | 3.43 |
| Firmicutes | Ruminococcaceae | Ruminococcus | | 1.85 | 2.11 | 4.7 | 2.11 | 1.49 | 4.73 |
| Firmicutes | Lachnospiraceae | Dorea | | 1.88 | 2.8 | 1.77 | 2.15 | 2.76 | 2.13 |
| Firmicutes | Ruminococcaceae | Other | Other | 5.54 | 3.82 | 2.47 | 1.85 | 0.62 | 3.46 |
| Firmicutes | Erysipelotrichaceae | [symbol] | | 0.63 | 0.38 | 2.25 | 1.18 | 2.4 | 2.88 |
| Proteobacteria | Desulfovibrionaceae | Bilophila | | 0.02 | 0.01 | 0.01 | 0.01 | 0.11 | 0.02 |
| Proteobacteria | Enterobacteriaceae | | | 0.66 | 0.74 | 0.33 | 9.8 | 0.95 | 0.08 |
| Proteobacteria | Alcaligenacene | Sutterella | | 0.04 | 0.06 | 0.01 | 0 | 0.01 | 0 |
| Verrucomicrobia | Verrucomicrobiaceae | Akkermansia | [symbol] | 0.17 | 0.12 | 1.49 | 0.13 | 0.72 | 0.91 |
| Tenericutes | | | | 0.19 | 0.12 | 0.2 | 0.08 | 0.01 | 0.21 |
| TM [symbol] | | | | 0 | 0 | 0.01 | 0.01 | 0.02 | 0.01 |
| Cyanobacteria | | | | 0.01 | 0.01 | 0.01 | 0.01 | 0.03 | 0.02 |

[symbol]

indicates data missing or illegible when filed

TABLE 3
Average relative species compositions of gut microbial communities for the
intervention groups 7 mg DCL, 7 mg GAL-MSFA and DC at the beginning and at the end of
the intervention study.

| | | | | | | 7 mg | | | |
|---|---|---|---|---|---|---|---|---|---|
| | | | | | | GAL- | 7 mg_GAL- | DC | DC |
| | | | | 7 mg_DCL | 7 mg_DCL | MSPA | MSFA | V_ | V_ |
| | | | | I_Day_0 | I_Week_4 | II_Day_0 | II_Week_4 | Day_0 | Week_4 |
| Bacteroidetes | Bacteroidaceae | Bacteroides | Other | 0.08 | 0.21 | 0.15 | 0.04 | 0.12 | 0.04 |
| Bacteroidetes | Bacteroidaceae | Bacteroides | uniformis | 1.71 | 0.41 | 0.24 | 0.03 | 0.21 | 0.07 |
| Bacteroidetes | Bacteroidaceae | Bacteroides | | 7.54 | 1.84 | 8.60 | 2.57 | 1.64 | 0.57 |
| Bacteroidetes | Bacteroidaceae | Bacteroides | eggerthii | 1.43 | 0.17 | 0.01 | 0.00 | 0.30 | 0.00 |
| Bacteroidetes | Prevotellaceae | Prevotella | copri | 2.05 | 22.30 | 13.55 | 12.19 | 0.28 | 2.72 |
| Bacteroidetes | [Odoribacteraceae) | Butyricimonas | | 0.11 | 0.01 | 0.08 | 0.03 | 0.06 | 0.01 |
| Bacteroidetes | Porphyromonadaceae | Parabacteroides | distasonis | 0.46 | 0.07 | 0.36 | 0.09 | 0.13 | 0.05 |
| Bacteroidetes | Porphyromonadaceae | Parabacteroides | | 0.48 | 0.05 | 0.33 | 0.10 | 0.26 | 0.32 |
| TM [symbol] | | | | 0.02 | 0.00 | 0.01 | 0.00 | 0.00 | 0.01 |
| Bacteroidetes | S24-7 | | | 0.00 | 0.00 | 0.72 | 1.71 | 1.55 | 1.56 |
| Bacteroidetes | [Paraprevotellaceae] | Paraprevotella | | 0.58 | 0.05 | 0.11 | 0.05 | 0.04 | 0.00 |
| Bacteroidetes | Bacteroidaceae | Bacteroides | [symbol] | 0.08 | 0.03 | 0.08 | 0.04 | 0.09 | 0.00 |
| Bacteroidetes | Bacteroidaceae | Bacteroides | [symbol] | 0.09 | 0.01 | 0.09 | 0.01 | 0.04 | 0.01 |
| Bacteroidetes | [Barnestellaceae] | | | 0.22 | 0.06 | 0.70 | 0.07 | 0.03 | 0.01 |
| Bacteroidetes | Prevotellaceae | Prevotella | [symbol] | 0.02 | 0.00 | 1.25 | 0.52 | 0.09 | 0.13 |
| Bacteroidetes | Prevotellaceae | Prevotella | | 0.42 | 0.00 | 0.27 | 0.19 | 0.08 | 0.64 |
| Bacteroideles | Rikenellaceae | | | 1.12 | 0.58 | 2.18 | 0.57 | 0.38 | 0.08 |
| Actinobacteria | Coriobacteriaceae | Collinsella | aerofaciens | 0.47 | 0.70 | 0.95 | 0.57 | 0.24 | 0.57 |
| Actinobacteria | Coriobacteriaceae | Slackia | | 0.02 | 0.06 | 0.09 | 0.09 | 0.07 | 0.15 |
| Actinobacteria | Bifidobacteriaceae | Bifidobacterium | adolescentis | 0.06 | 0.62 | 0.33 | 1.09 | 1.13 | 0.58 |
| Actinobacteria | Bifidobacteriaceae | Bifidobacterium | longum | 0.37 | 0.83 | 0.09 | 0.11 | 0.23 | 0.08 |
| Actinobacteria | Bifidobacteriaceae | Bifidobacterium | | 0.66 | 0.42 | 0.16 | 0.54 | 1.29 | 0.41 |
| Actinobacteria | Coriobacteriaceae | | | 0.20 | 0.52 | 0.83 | 0.41 | 1.36 | 1.50 |
| Actinobacteria | Coriobacteriaceae | Adlercreutzia | | 0.03 | 0.03 | 0.02 | 0.01 | 0.03 | 0.05 |
| Actinobacteria | Coriobacteriaceae | Atopobium | | 0.01 | 0.01 | 0.01 | 0.01 | 0.01 | 0.02 |
| Actinobacteria | [symbol] | Actinomyces | | 0.03 | 0.04 | 0.02 | 0.01 | 0.02 | 0.04 |
| Firmicutes | Ruminococcaceae | Ruminococcus | | 1.52 | 1.01 | 1.86 | 2.11 | 1.45 | 1.92 |
| Firmicutes | Clostridiaceae | Clostridium | | 0.50 | 0.29 | 0.32 | 0.45 | 0.61 | 0.75 |
| Firmicutes | Lachnospiraceae | Shuttleworthia | | 0.10 | 0.12 | 0.12 | 0.10 | 0.07 | 0.09 |
| Firmicutes | | | | 7.10 | 7.06 | 6.98 | 6.65 | 5.43 | 5.16 |
| Firmicutes | Lachnospiraceae | Coprococcus | | 2.19 | 2.06 | 1.78 | 2.06 | 1.66 | 1.76 |
| Firmicutes | Ruminococcaceae | Other | | 0.83 | 1.10 | 5.54 | 3.82 | 0.87 | 0.54 |
| Firmicutes | Clostridiaceae | | | 1.88 | 0.99 | 0.95 | 0.75 | 0.58 | 0.94 |
| Firmicutes | Lachnospiraceae | Blautia | | 1.22 | 1.61 | 0.87 | 0.68 | 1.37 | 1.49 |
| Firmicutes | Christensenellaceae | | | 0.51 | 0.52 | 0.23 | 0.27 | 0.36 | 1.61 |
| Firmicutes | Other | Other | | 1.55 | 1.29 | 1.31 | 0.78 | 1.06 | 1.22 |
| Firmicutes | Erysipelotrichaceae | Catenibacterium | | 0.10 | 0.79 | 0.63 | 0.38 | 0.83 | 1.11 |
| Firmicutes | Lachnospiraceae | Roseburia | | 0.10 | 0.05 | 0.08 | 0.14 | 0.20 | 0.25 |
| Firmicutes | Ruminococcaceae | Ruminococcus | flavefaciens | 0.22 | 0.10 | 0.07 | 0.03 | 0.00 | 0.02 |
| Firmicutes | Lachnospiraceae | Blautia | | 3.78 | 5.86 | 3.08 | 2.24 | 4.21 | 4.65 |
| Firmicutes | Veillonellaceae | Dialister | | 1.08 | 1.00 | 0.30 | 0.82 | 3.64 | 1.25 |
| Firmicutes | Ruminococcaceae | Faecalibacterium | [symbol] | 2.21 | 3.35 | 1.39 | 2.43 | 2.00 | 2.34 |
| Firmicutes | Ruminococcaceae | Oscillospira | | 1.22 | 1.24 | 1.71 | 1.44 | 2.63 | 1.02 |
| Firmicutes | Erysipelotrichaceae | | | 0.06 | 0.06 | 0.08 | 0.04 | 0.06 | 0.02 |
| Firmicutes | Streptococcaceae | Streptococcus | | 0.36 | 0.59 | 0.13 | 0.21 | 0.26 | 3.29 |
| Firmicutes | Lactobacillaceae | Lactobacillus | | 0.02 | 0.01 | 0.00 | 0.02 | 0.02 | 0.14 |
| Firmicutes | Ruminococcaceae | | | 20.67 | 19.42 | 20.96 | 30.93 | 33.04 | 26.33 |
| Firmicutes | Lachnospiraceae | [Ruminococcus] | Other | 0.01 | 0.01 | 0.01 | 0.01 | 0.01 | 0.02 |
| Firmicutes | [Mogibacteriaceae] | | | 0.04 | 0.05 | 0.18 | 0.11 | 0.09 | 0.11 |
| Firmicutes | Lachnospiraceae | [Ruminococcus] | [symbol] | 0.06 | 0.04 | 0.04 | 0.04 | 0.05 | 0.07 |
| Firmicutes | Erysipelotrichaceae | Bulleidia | p-1630- [symbol] | 0.01 | 0.00 | 0.59 | 0.09 | 0.08 | 0.07 |
| Firmicutes | Lachnospiraceae | [symbol] | | 0.04 | 0.04 | 0.04 | 0.02 | 0.05 | 0.09 |
| Firmicutes | Lachnospiraceae | Dorea | | 3.64 | 2.31 | 1.87 | 2.81 | 1.61 | 3.00 |
| Firmicutes | Veillonellaceae | Veillonella | dispar | 0.04 | 0.05 | 0.01 | 0.03 | 0.16 | 0.27 |
| Firmicutes | Veillonellaceae | Phascolarctobacterium | | 0.22 | 0.12 | 0.39 | 0.17 | 0.08 | 0.01 |
| Firmicutes | Lachnospiraceae | [Ruminococcus] | | 0.27 | 0.17 | 0.20 | 0.30 | 0.24 | 0.41 |
| Firmicutes | Clostridiaceae | Other | Other | 0.06 | 0.03 | 0.03 | 0.01 | 0.01 | 0.01 |
| Firmicutes | Lachnospiraceae | Coprococcus | Other | 0.53 | 0.37 | 0.13 | 0.22 | 0.21 | 0.25 |
| Firmicutes | Lachnospiraceae | [symbol] | | 0.73 | 0.36 | 0.25 | 0.11 | 0.44 | 0.84 |
| Firmicutes | Lachnospiraceae | Other | Other | 7.50 | 5.93 | 4.84 | 5.90 | 6.08 | 10.35 |
| Firmicutes | Erysipelotrichaceae | [Eubacterium] | biforme | 0.14 | 0.13 | 0.53 | 0.27 | 0.09 | 0.32 |
| Firmicutes | Lachnospiraceae | | | 11.09 | 9.48 | 7.01 | 8.05 | 6.73 | 9.18 |
| Proteobacteria | Enterobacteriaceae | | | 0.35 | 0.08 | 0.67 | 0.74 | 2.51 | 0.65 |
| Protobacteria | Alcaligenaceae | Sutterella | | 0.07 | 0.04 | 0.04 | 0.06 | 0.03 | 0.19 |
| Promobacteria | Desulfovibrionaceae | Bilophila | | 0.01 | 0.01 | 0.02 | 0.01 | 0.02 | 0.01 |
| Tenericutes | | | | 0.14 | 8.16 | 0.18 | 0.12 | 0.40 | 1.44 |
| Verrucomicrobia | Verrucomicrobiaceae | Akkermansia | muciniphila | 2.02 | 2.68 | 0.17 | 0.12 | 5.41 | 4.58 |

[symbol]

indicates data missing or illegible when filed

Blood and Tissue Parameters

Ingestion of lycopene products for one month, either in the capsule format or in the chocolate matrix, resulted in a significant increase of its concentration both in the serum and in the ear skin excretion (Table 4).

TABLE 4
Changes in blood and tissue parameters after supplementation with GA
lycopene for one month.

| Parameters before | | | | | |
|---|---|---|---|---|---|
| and after 4 weeks | Groups | | | | |
| of the trial | I | II | III | IV | V |
| Lycopene in | | | | | |
| serum, in ng/ml | | | | | |
| before | 110 ± 17 | 110 ± 12 | 210 ± 19 | 90 ± 8.4 | 120 ± 22 |
| after | 500 ± 52** | 310 ± 30** | 430 ± 30** | 190 ± 14* | 170 ± 27 |
| Lycopene in | | | | | |
| cerumen, in ng/g | | | | | |
| before | 53 ± 9.5 | 40 ± 5.5 | 70 ± 10.2 | 750 ± 93 | 14 ± 7.6 |
| after | 102 ± 12.4* | 100 ± 12.5* | 90 ± 11.5 | 2,500 ± 237** | 12 ± 5.5 |
| Triglycerides | | | | | |
| mg/dL | | | | | |
| before | 135 ± 14.9 | 155 ± 12.1 | 128 ± 9.7 | 126 ± 10.2 | 122 ± 13.5 |
| after | 133 ± 11.5 | 150 ± 11.3 | 110 ± 8.5* | 123 ± 10.1 | 118 ± 11.7 |
| LDL, in mg/dL | | | | | |
| before | 144 ± 12.5 | 143 ± 12.4 | 121 ± 10.5 | 137 ± 11.7 | 131 ± 12.1 |
| after | 139 ± 10.1* | 134 ± 11.2* | 104 ± 9.8* | 124 ± 10.3* | 129 ± 10.2 |
| HDL, in mg/dL | | | | | |
| before | 41.9 ± 2.9 | 46.5 ± 3.7 | 49.8 ± 3.9 | 50.1 ± 4.2 | 44.0 ± 2.2 |
| after | 42.2 ± 3.1 | 47.8 ± 3.9 | 50.0 ± 4.6 | 51.2 ± 4.4 | 45.1 ± 2.4 |
| IOD, in μM MDA | | | | | |
| before | 142 ± 9.2 | 141 ± 12.7 | 115 ± 10.9 | 64 ± 5.8 | 177 ± 12.1 |
| after | 101 ± 8.7** | 92 ± 8.8** | 46 ± 4.5** | 42 ± 3.7* | 153 ± 11.9* |
| LDL-Px, in | | | | | |
| ELISA × 1O″ | | | | | |
| before | 310 ± 29 | 550 ± 61 | 664 ± 63 | 420 ± 45 | 450 ± 41 |
| after | 250 ± 24* | 350 ± 29** | 379 ± 34** | 130 ± 12** | 370 ± 32* |
| Lipoprotein 0₂, | | | | | |
| in μM | | | | | |
| before | 4.07 ± 0.29 | 3.89 ± 0.35 | 3.86 ± 0.32 | 3.07 ± 0.29 | 3.67 ± 0.31 |
| after | 5.26 ± 0.33* | 4.64 ± 0.33* | 4.55 ± 0.39* | 3.44 ± 0.27 | 5.27 ± 0.39* |
| StO₂, in AUC | | | | | |
| mm | | | | | |
| before | 81 ± 6.4 | 66 ± 5.2 | 67 ± 5.1 | 59 ± 4.4 | 76 ± 5.5 |
| after | 88 ± 6.9* | 79 ± 6.1* | 83 ± 7.1* | 79 ± 6.3* | 76 ± 6.3 |

*p < 0.05,
**p < 0.001

Supplementation with GAL-MSFA resulted in a dose-depended significant reduction of markers of oxidative damage and inflammation. 7 mg of lycopene was able to reduce IOD and LDL-Px, by the end of the month, by 49 μM MDA and by 200 ELISA units, whilst 30 mg reduced these parameters by 69 and 285, accordingly. 30 mg of GAL-MSFA was 3-fold more effective to inhibit IOD than the same dose of lycopene but in the GAL-PUFA formulation. This may potentially indicate the possible liver origin of this blood marker. Effect of two formulations of lycopene on LDL-Px was similar (Table 4)

DC with or without lycopene had a similar effect on the inhibition of IOD as 7 mg of lycopene. Although both chocolate products were able to reduce LDL-Px, their effectiveness was below that of lycopene itself.

Administration of either formulation of GAL, or lycopene with DC complex, resulted by significant changes in the profile of fasting lipoproteins, which are assembled and produced by the liver. GAL-MSFA reduced in a dose-dependent manner both LDL concentration and triglycerides. This liver-targeting formulation of lycopene, in 30 mg dose, was able to reduce the first parameter by 17 mg/dL and the second by 18 mg/dl. Supplementation with GAL-PUFA resulted in LDL reduction by 13 mg/dL and triglycerides by only 3 mg/dl. Lycopene in the L-tug complex with dark chocolate was also able to reduce LDL, however, changes caused by the ingestion of the control DC were not significant (Table 4). By the end of the trial there were no changes in the serum concentration of HDL, glucose and liver enzymes, ALT and AST (results are not presented).

There were noticeable improvements in the molecular oxygen metabolism in all groups. In groups supplemented with GAL-MSFA 02 concentration and its transportation by blood lipoproteins was increased by 18-19%. In the group that received GAL-PUFA this increase was lower, by 12%. In the group, which received control DC the increase in the lipoprotein 02 was the highest, by 44%. These changes in the plasma oxygen transportation translated to benefit peripheral tissue oxygenation but not in the control DC group. Ingestion of all lycopene products significantly boosted tissue oxygenation in skeletal muscles. Administration of GAL-MSFA demonstrated a dose dependent effect in changes of this parameter. However, 30 mg of lycopene in GAL-PUFA formulation was 25% more effective than the same dose of lycopene but in the GAL-MSFA formulation (Table 4).

Development of the metabolic syndrome and ageing are accompanied by ongoing, often at a subclinical level, processes of inflammatory and oxidative damage which may lead to changes in the liver metabolism, vascular functions, increase body mass and development subclinical systemic tissue hypoxia. In this study supplementation with lycopene, especially formulated for effective bioavailability in middle-aged people, was observed to have antioxidant, anti-inflammatory and lipid-lowering effects which are in accordance with earlier reports [Petyaev, 2012]. These changes in the blood markers were accompanied, or maybe resulted, in the improvement in the peripheral tissue StO₂. The main contributor to this parameter was the skeletal muscle respiration, although skin oxygenation may be part of it too.

There are a number of molecules within the food, which are not fully digestible; hence they can reach the colon and its microbiota. Carotenoids and lycopene in particular belong to these types of partially digestible molecules. In this study, regular intake by moderately obese middle-aged persons of 7 or 30 mg of GA lycopene, whether in a capsule form or in a dark chocolate matrix, for one month was observed to result in a significant change in the profile of the gut microbiota, and in particular an increase of the population of Bifidobacteria adolescentis. Bifidobacteria probiotics is one of the best-known probiotics, which has a broad range of health beneficial properties not only in the gut environment but in the whole body. This involves their ability to control bacterial and viral pathogens, stimulate local intestinal and systemic immune system, improve lipid metabolism and weight management [Servin, 2004 and Amy O'Callaghan]. The loss of the Bifidobacteria could be a significant factor associated with ageing [Arboleya, 2016]. Therefore, the observed changes in the inflammatory markers, blood lipids and age-associated changes in the tissue oxygenation and other parameters may not only be the result of lycopene accumulation in the body but may also be a result of the restoration of the population of Bifidobacteria adolescentis in the gut.

Observed lycopene effects on the gut bacteria, blood markers of inflammation and oxidation, lipids produced by the liver and by the skin (sebum) and peripheral tissue oxygenation were all dose-dependent. This indicates either a possible direct and parallel involvement of lycopene in affecting such different targets in the body independently, or there is “a bridge”, signaling and/or metabolic, which closely interconnect the gut bacteria, and in particular Bifidobacteria adolescentis population, with the blood, liver and other tissues, which lycopene can control.

Lycopene molecules directly affected growth of the Bifidobacteria adolescentis and/or their inclusion in the intestine and the colon. The improvement of metabolism and physiology of the gut tissues may lead to better control of the microbiota and boost growth of health beneficial bacteria.

To conclude, the observed systemic effect of lycopene supplementation improved gut, blood, liver lipid metabolism may not be due to the carotenoid compound and dark chocolate themselves but may also be due to their ability to be effective prebiotics for the Bifidobacteria.

### REFERENCES

Castes F, Denis C, Roche F, Prieur F, Enjolras F, Barthelemy J C. “Age-associated alteration of muscle oxygenation measured by near infrared spectroscopy during exercise.” Arch Physiol Biochem. (1999) April; 107(2): 159-67.
Lucia Marseglia, Sara Manti, Gabriella D'Angelo, Antonio Nicotera, Eleonora Parisi, Gabriella Di Rosa, Eloisa Gitto and Teresa Arrigo “Oxidative Stress in Obesity: A Critical Component in Human Diseases” Int. J. Mot Sci. (2015) 16, 378-400.
Ovreas L, Forney L, Daae F L, and Torsvik V. “Distribution of bacterioplankton in meromictic Lake Saelenvannet, as determined by denaturing gradient gel electrophoresis of PCR-amplified gene fragments coding for 16S rRNA.” Applied and environmental microbiology (1997) 63:3367-3373.
Kristensen K H S, Wiese M, Rytter M J H, 6z9 am M, Hansen LH, Namusoke H, Friis H, and Nielsen D S. “Gut microbiota in children hospitalized with oedematous and non-oedematous severe acute malnutrition in Uganda.” PLoS Negl Trap Dis (2016) 10:e0004369.
Edgar R C. “UPARSE: highly accurate OTU sequences from microbial amplicon reads.” Nature methods (2013) 10:996-998.
Caporaso J G, Kuczynski J, Stombaugh J, Bittinger K, Bushman F D, Costello E K, Fierer N, Pena A G, Goodrich J K, and Gordon J I. “QIIME allows analysis of high-throughput community sequencing data.” Nature methods (2010) 7:335-336.
McDonald D, Price M N, Goodrich J, Nawrocki E P, Desantis T Z, Probst A, Andersen G L, Knight R, and Hugenholtz P. An improved Greengenes taxonomy with explicit ranks for ecological and evolutionary analyses of bacteria and archaea.” The ISME Journal (2012) 6:610-618.
Petyaev I. M., Mitchinson M. J., Hunt J. V., Coussons P. J. “Superoxide dismutase activity of antibodies purified from the human arteries and atherosclerotic lesions.” Biochem.Soc.Trans. (1998) v.26, S43-45.
Ivan M. Petyaev, Pavel Y. Dovgalevsky, Victor A. Klochkov, Natalya E. Chalyk, and Nigel Kyle “Clinical Study: Whey Protein Lycosome Formulation Improves Vascular Functions and Plasma Lipids with Reduction of Markers of Inflammation and Oxidative Stress in Prehypertension.” The Scientific World Journal” (2012) article ID 269476, doi:10.1100
Servin A L. “Antagonistic activities of Lactobacilli and bifidobacteria against microbial pathogens.” FEMS Microbial Rev. (2004) October; 28(4):405-440.
Amy O'Callaghan and Douwe van Sinderen “Bifidobacteria and Their Role as Members of the Human Gut Microbiota” Front Microbial (2016) v. 7, doi: 10.3389/fmicb.2016.00925.
Arboleya S, Watkins C, Stanton C, Ross RP. “Gut Bifidobacteria Populations in Human Health and Aging.” Front Microbial (2016) 19, 7, 1204. doi: 10.3389/fmicb.2016.01204.
Maria Wiese, Yuriy Bashmakov, Natalia Chalyk, Dennis Sandris Nielsen, Aukasz Krych, Witold Kot, Victor Klochkov, Dmitry Pristensky, Tatyana Bandaletova, Marina Chernyshova, Nigel Kyle, and Ivan Petyaev “Prebiotic Effect of Lycopene and Dark Chocolate on Gut Microbiome with Systemic Changes in Liver Metabolism, Skeletal Muscles and Skin in Moderately Obese Persons” Hindawi BioMed Research International (2019): 1-15, https://doi.org/10.1155/2019/4625279

1. A method for inhibiting growth or decreasing the abundance of Gram-negative anaerobic pathogenic bacteria in the gastro-intestinal tract, mouth, skin, respiratory or urogenital system of a subject in need thereof, the method comprising administering to the subject a therapeutically effective amount of a composition comprising a carotenoid compound, wherein the bacteria is of the genera Desulfovibrio, Prevotella and/or Dialister.

2. The method according to claim 1, wherein the carotenoid compound is selected from the group consisting of lycopene, lutein, zeaxanthin, meso-zeaxanthin, astaxanthin, α- or β-carotenes, cryptoxanthin, flavoxanthin, neoxanthin, or another tetraterpenoid.

3. The method according to claim 2, wherein the carotenoid compound is a lycopene.

4. The method according to claim 1, for prophylactic treatment of a condition associated with the status of the gastro-intestinal tract, mouth, skin, respiratory or urogenital system health.

5. The method according to claim 4, wherein the condition is selected from the group consisting of gastrointestinal diseases, inflammatory bowel syndrome, constipation, diarrhoea, colitis, Crohn's disease, appendicitis, ulcerated colitis, functional bowel disorder, irritable bowel syndrome, periodontitis, gingivitis, other mouth pathologies, skin diseases, dermatitis, psoriasis, neurodegenerative diseases, Parkinson's disease, Alzheimer's disease, other forms of dementia, multiple sclerosis, colon cancer, breast cancer, other forms of cancer, urogenital system pathologies, other disorders associated with growth or inflammation associated with or caused by these bacteria or their metabolites, including metabolic syndrome, obesity, cardio- and cerebrovascular diseases, mental pathologies, or ageing processes.

6. The method according to claim 1, further comprising administering one or more probiotic bacteria.

7. The method according to claim 6, wherein the one or more probiotic bacteria is selected from the group consisting of Lactobacillus plantarum, Lactobacillus acidophilus, Lactobacillus paracasei, Leuconostoc mesenteroides, Lactobacillus bulgaricus, Lactobacillus casei, Lactobacillus salivarius, Acidophilus, Acidophilus Bifidus, Acidophilus Lactobacillus, L. Acidophilus, L. Amylovorus, L. Brevis, L. Casei Immunitas, L. Crispatus, L. Delbrueckii, L. Fermentum, L. Gallinarum, L. Helveticus, L. Johnsonii, L. Johnsonii LC-1, L. Lactis, L. Plantarum, L. Reuteri, L. Rhamnosus, L. Salivarius, L. Sporogenes, Lacto Bacillus, Lactobacille, Lactobacilli, Lactobacilli Bulgaricus, Lactobacilli Plantarum, Lactobacilli Rhamnosus, Lactobacilli Salivarium, Lactobacillus acidophilus, Lactobacillus amylovorus, Lactobacillus brevis, Lactobacillus bulgaricus, Lactobacillus casei, Lactobacillus casei sp. rhamnosus, Lactobacillus crispatus, Lactobacillus delbrueckii, Lactobacillus delbrueckii ssp. bulgaricus, Lactobacillus fermentum, Lactobacillus gallinarum, Lactobacillus Gasseri, Lactobacillus GG, Lactobacillus Helveticus, Lactobacillus johnsonii, Lactobacillus Lactis, Lactobacillus reuteri, Lactobacillus Rhamnosus GG, Lactobacillus rhamnosus, Lactobacillus sakei, Lactobacillus sporogenes, Lactobacilo, Lactospores, LC-1, Probiotics, Probiotiques, Pediococcus pentosaceus, Streptococcus thermophilus, Bacillus subtilis, Bacillus coagulans, Enteroccous faecium, Bifidobacterium bifidum, Bifidobacterium lactis, Bifidobacterium Longum, and Bifidobacterium infantis, Bifidobacterium breve, Bifidobacterium animalis or Bifidobacterium longum.

8. The method according to claim 1, wherein the composition is a nutraceutical or liquiceutical, or pharmaceutical composition, or an ingredient of functional food or beverages.

9. The method according to claim 8, wherein the composition comprises at least about 30% w/w of the carotenoid.

10. The method according to claim 9, wherein the composition comprises at least about 50% w/w of the carotenoid.
